(19) United States
(12) Patent Application Publication
(10) Pub. No.: US 2005/0025778 A1
(43) Pub. Date: Feb. 3, 2005

(54) Microprojection array immunization patch and method

(76) Inventors: Michel J.N. Cormier, Mountain View, CA (US); James Matriano, Mountain View, CA (US); Juanita A. Johnson, Belmont, CA (US); Wendy A. Young, Cupertino, CA (US); Peter E. Daddona, Menlo Park, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/884,603

(22) Filed: Jul. 1, 2004

Related U.S. Application Data
(60) Provisional application No. 60/484,930, filed on Jul. 2, 2003.

(51) Int. Cl. A61K 39/00

(52) U.S. Cl. 424/185.1

(57) ABSTRACT

Microprojection members (10) having a reservoir containing an antigenic agent and methods of using such members to vaccinate mammals (e.g., humans) are disclosed. The microprojection members are used to transdermally deliver an antigenic agent (e.g., a vaccine antigen) with substantially reduced skin reactions. This is achieved by delivering an induction amount and thereafter delivering one or more subsequent booster amounts. The induction amount is relatively larger than the booster amount. This technology has broad applicability for a wide variety of therapeutic vaccines to improve efficacy and convenience of use.

### CROSS-REFERENCE TO RELATED APPLICATIONS

This application claims the benefit of U.S. Provisional Application No. 60/484,930, filed Jul. 2, 2003.

### FIELD OF THE PRESENT INVENTION

The present invention relates generally to active agent delivery systems and methods. More particularly, the invention relates to transdermal delivery of antigenic agents via microprojection arrays.

### BACKGROUND ART

It is well known that delivery or administration of an antigenic agent, such as a vaccine, can be achieved through various routes of administration, including oral, nasal, intramuscular (IM), subcutaneous (SC), and intradermal (ID). It is further well documented that the route of administration can impact the type of immune response. See, for example, LeClerc, et al., “Antibody Response to a Foreign Epitope Expressed at the Surface of Recombinant Bacteria: Importance of the Route of Immunization,” Vaccine, vol. 7, pp. 242-248 (1989).

The majority of commercial vaccines are administered by IM or SC routes. In almost all cases, they are administered by conventional injection with a syringe and needle, although high velocity liquid jet-injectors have had some success. See, for example, Parent du Chatelet, et al., Vaccine, Vol. 15, pp. 449-458 (1997).

As an alternative to the more conventional routes of administration, increasing interest is being placed on ID routes of delivery to capitalize on the skin's function as an immune organ. See, for example, Tang, et al., Nature, vol. 388, pp. 729-730 (1997); Fan, et al., Nature Biotechnology, vol. 17, pp. 870-872 (1999); and Bos, J. D., ed., Skin Immune System (SIS), Cutaneous Immunology and Clinical Immunodermatology, CRC Press, pp. 43-146 (2^{nd} Ed., 1997).

Pathogens entering the skin are confronted with a highly organized and diverse population of specialized cells that are capable of eliminating microorganisms through a variety of mechanisms. Epidermal Langerhans cells (LC) are potent antigen-presenting cells found in the viable epidermis. Lymphocytes and dermal macrophages percolate throughout the dermis and form a semi-continuous network. Keratinocytes and Langerhans cells express or can be induced to generate a diverse array of immunologically active compounds. Collectively, these cells orchestrate a complex series of events that ultimately control both innate and specific immune responses.

The normal function of the LC's is to detect, capture and present antigens to evoke an immune response to invading pathogens. LC's perform this function by internalizing epicutaneous antigens, trafficking to regional skin-draining lymph nodes, and presenting processed antigens to T cells. A discussion of the skin's role in the immune system can be found in Fichtelius, et al., Int. Arch. Allergy, vol. 37, pp. 607-620 (1970), and Sauder, J., Invest. Dermatol, vol. 95, pp. 105-107 (1990).

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

Despite these advantages, practical, reliable, and minimally invasive methods for delivering antigens specifically into the epidermis and/or dermis in humans are still under development. A significant limitation to intradermal injection is that the use of conventional needles requires a very high level of eye-hand coordination and finger dexterity. Accordingly, there has been a growing interest in the development of needle-free vaccine delivery systems.

Independent laboratories have demonstrated needle-free immunization to macromolecules, including protein- and DNA-based antigens. Glenn, et al. demonstrated that a solution containing tetanus toxoid mixed with an adjuvant, cholera toxin, applied on untreated skin is capable of inducing anti-cholera toxin antibodies. Glenn, et al., Nature, vol. 391, p. 851 (1998).

Tang, et al. further demonstrated that topical administration of an adenoviral vector encoding human carcinoembryonic antigen induces antigen-specific antibodies. Tang, et al., Nature, vol. 388, pp. 729-730 (1997). Fan, et al. also demonstrated that topical application of naked DNA encoding for hepatitis B surface antigen can induce cellular and humoral immune responses. Fan, et al., Nature Biotechnology, vol. 17, pp. 870-872 (1999).

Accordingly, transdermal delivery provides for a method of administering antigenic agents that would otherwise need to be delivered via hypodermic injection, intravenous infusion or orally. Transdermal vaccine delivery offers improvements in both of these areas. Transdermal delivery when compared to oral delivery avoids the harsh environment of the digestive tract, bypasses gastrointestinal drug metabolism, reduces first-pass effects, and avoids the possible deactivation by digestive and liver enzymes. Conversely, the digestive tract is not subjected to the vaccine during transdermal administration.

The word “transdermal”, as used herein, is generic term that refers to delivery of an antigentic agent (e.g., a vaccine or other immunologically active agent) through the skin to the local tissue, particularly the dermis and epidermis, or systemic circulatory system without substantial cutting or penetration of the skin, such as cutting with a surgical knife or piercing the skin with a hypodermic needle. Transdermal agent delivery includes delivery via passive diffusion as well as active delivery based on external energy sources such as electrical (iontophoresis, for example) and ultrasound (phonophoresis, for example).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, passive transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum, particularly to hydrophilic and high molecular weight drugs and macromolecules such as proteins, naked DNA, and viral vectors. Consequently, transdermal delivery has been generally limited to the passive delivery of low molecular weight compounds (<500 daltons) with limited hydrophilicity. This generally does not allow delivery of immunologically effective amounts of an antigenic agent.

One common method of increasing the passive transdermal diffusional agent flux involves pre-treating the skin with or co-delivering with the agent, a skin permeation enhancer, such as chemical permeation enhancers, depilatories, occlusion, and hydration techniques that increase permeability to macromolecules. However, these methods may not be able to deliver therapeutic doses without prolonged wearing times, and they can be relatively inefficient means of delivery. Furthermore, the effects of chemical permeation enhancers are limited at nonirritating concentrations. The efficacy of these methods in enhancing transdermal flux has also been limited for the larger proteins, primarily due to their size.

There also have been many techniques and systems developed to mechanically penetrate or disrupt the outermost skin layers thereby creating pathways into the skin in order to enhance the amount of agent being transdermally delivered. Such physical methods of permeation enhancement include sandpaper abrasion, tape stripping, and bifurcated needles. While these techniques increase permeability, it is difficult to predict the magnitude of their effect on drug absorption. Laser ablation, another physical permeation enhancer, may provide more reproducible effects, but it is currently cumbersome and expensive.

Early vaccination devices, known as scarifiers, generally included a plurality of tines or needles that were applied to the skin to and scratch or make small cuts in the area of application. The vaccine was applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

However, a serious disadvantage in using a scarifier to deliver an active agent, such as a vaccine, is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Additionally, due to the self-healing process of the skin, the punctures or slits made in the skin tend to close up after removal of the piercing elements from the stratum corneum. Thus, the elastic nature of the skin acts to remove the active agent liquid coating that has been applied to the tiny piercing elements upon penetration of these elements into the skin. Furthermore, the tiny slits formed by the piercing elements heal quickly after removal of the device, thus limiting the passage of the liquid agent solution through the passageways created by the piercing elements and in turn limiting the transdermal flux of such devices.

Other systems and apparatus that employ tiny skin piercing elements to enhance transdermal agent delivery are disclosed in U.S. Pat. Nos. 5,879,326, 3,814,097, 5,250,023, 3,964,482, U.S. Pat. Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all of which are incorporated herein by reference in their entirety.

These prior art systems employ piercing elements of various shapes and sizes to pierce the outermost layer (i.e., the stratum corneum) of the skin. The piercing elements, or microprojections, disclosed in these references generally extend perpendicularly from a thin, flat member, such as a pad or sheet. Generally, a plurality of microprojections are arranged in an array to provide a transdermal delivery patch. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

Microprojection array patch technology is accordingly being developed to increase the number of type of agents that can be transdermally delivered through the skin. Upon application, the microprojections create superficial pathways through the transport barrier of the skin (stratum corneum) to facilitate hydrophilic and macromolecule delivery. When delivering antigenic agents (e.g., vaccine antigens) intradermally via microprojection arrays, skin reactions have been found to be minimal following the primary immunization. Nevertheless, there remains a need to minimize skin reactions including local redness and edema following booster administration.

Accordingly, it is an object of the invention to provide a method of vaccinating a mammal by transdermally delivering an antigenic agent using microprojections.

It is a further object of the invention to transdermally deliver an antigenic agent with a plurality of administrations.

It is yet another object of the invention to minimize skin reactions to a transdermally delivered vaccination.

### SUMMARY OF THE INVENTION

In accordance with the above objects and those that will be mentioned and will become apparent below, the delivery member or immunization patch for transdermally delivering an antigenic agent, such as a vaccine, in accordance with this invention, includes a microprojection array and a reservoir adapted to receive at least one antigenic agent. The microprojection array comprises a plurality of skin-piercing microprojections that are adapted to make cuts through the outermost layer (i.e., the stratum cornea layer) of the skin and to penetrate into the underlying epidermis and/or dermis layers of the skin. Preferably, the microprojections do not pierce so deeply as to reach the capillary beds and cause significant bleeding.

In one embodiment of the invention, the delivery member has a microprojection density of at least approximately 10 microprojections/cm², more preferably, in the range of at least approximately 200-2000 microprojections/cm². In other embodiments, the delivery member includes a single microprojection.

In one embodiment, the delivery member is constructed out of stainless steel, titanium, nickel titanium alloys, or similar biocompatible materials.

In an alternative embodiment, the delivery member is constructed out of a non-conductive material, such as a polymer. Alternatively, the delivery member can be coated with a non-conductive material, such as Parylene®.

In accordance with one embodiment of the invention, the method for delivering an antigenic agent to a host or mammal (i.e., vaccination) comprises providing a delivery system having at least two transdermal delivery members, each transdermal delivery member having a plurality of microprojections (or arrays thereof) configured to pierce the stratum corneum and a reservoir adapted to receive an antigenic agent, the reservoir being positioned in antigenic agent-transmitting relation with the mammal, delivering with a first transdermal delivery member an induction amount of the antigenic agent, and at least about 7 days thereafter, delivering with a second transdermal delivery member a booster amount of the antigenic agent, the booster amount being up to about 50% by weight of the induction amount.

In at least one embodiment of the invention, the reservoir comprises a region of the delivery member that is positioned distal to but in communication with the microprojections. In other embodiments, the reservoir comprises a biocompatible coating that is disposed on the delivery member, preferably, on the microprojections. In yet other embodiments, the reservoir comprises a solid medium wherein the system further includes a hydration medium that is adapted to cooperate with the solid medium.

In accordance with the present invention, a relatively larger dose of the antigenic agent is delivered intradermally in a first application step via a first delivery member and thereafter one or more relatively smaller doses of antigenic agent are delivered intradermally via a second delivery member in one or more subsequent application steps. Typically, the amount of antigenic agent delivered in the subsequent application step(s) is less than about 50% by weight of the amount delivered in the first application step.

In accordance with one embodiment of the present invention, a delivery system comprising two delivery members having microprojection arrays of substantially the same size and construction are utilized in a two-step method. In the first dose administration, the microprojection array is left in skin-piercing contact with the mammal for a longer period of time compared to the period of contact time in the one or more subsequent dose administrations. In this manner, the first microprojection array delivers a larger dose of the antigenic agent than the subsequent administrations.

Preferably, when delivering the first dose of the antigenic agent, the microprojections are maintained in skin-piercing relationship to the skin of the host or mammal (e.g., a human patient) for at least about 0.5 hours, more preferably, at least about one hour, even more preferably, between one and twenty-four hours. When delivering the subsequent dose or doses of the antigenic agent, the microprojections are preferably maintained in skin-piercing relation with the skin for less than one hour, more preferably, less than 0.25 hours.

In accordance with a second embodiment of the present invention, the first microprojection array applied to the patient has a larger number of microprojections, a larger effective skin contact area and/or a higher concentration of antigenic agent in the reservoir compared to the subsequently applied microprojection arrays. In this way, the first applied microprojection array delivers a relatively higher dose of the antigenic agent than the subsequently applied microprojection arrays.

Preferably, the period of time between the first delivery member application and the second delivery member application is at least 7 days, more preferably, at least 14 days, even more preferably, at least about 21 days. Those skilled in the art will appreciate, however, that the period of time between the initial application and the subsequent booster applications will vary in large part with the particular antigenic agent being delivered as well as the age of the patient (e.g., child or adult).

Furthermore, the relative amounts of the antigenic agent delivered in the first application and the one or more subsequent booster applications will also be highly dependent upon the particular antigenic agent and its recommended dosage, as well as the age of the patient.

In accordance with the invention, the antigenic agent can comprise vaccines, including protein-based vaccines, polysaccharide-based vaccine and nucleic acid-based vaccines, viruses and bacteria.

Commercially available vaccines useful in the practice of the invention, which contain antigenic agents, include, without limitation, flu vaccines, lyme disease vaccine, rabies vaccine, measles vaccine, mumps vaccine, chicken pox vaccine, small pox vaccine, hepatitis vaccine, pertussis vaccine, and diphtheria vaccine.

Other suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include. DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitdis, pseudomonas aeruginosa, streptococcus pneumoniae, treponema pallidum, and vibrio cholerae, and mixtures thereof.

In some embodiments of the invention, the delivery system further includes a hydrogel. In the embodiments noted above wherein the reservoir is located distal to the microprojections, the antigenic agent is preferably formulated in the hydrogel. In alternative embodiments, the hydrogel does not contain the antigenic agent and, hence, functions as a hydration medium.

The hydrogel preferably comprises a water-based hydrogel having a macromolecular polymeric network. In a preferred embodiment of the invention, the polymer network comprises, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

The hydrogel and formulations thereof preferably includes one surfactant, which can be zwitterionic, amphoteric, cationic, anionic, or nonionic. Suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols such as laureth-4.

In further embodiments of the invention, the hydrogel formulation includes a polymeric material or polymer having amphiphilic properties, which can comprise, without limitation, cellulose derivatives, such as hydroxyethylcellulose (HEC), hydroxypropyl-methylcellulose (HPMC), hydroxypropycellulose (HPC), methylcellulose (MC), hydroxyethylmethylcellulose (HEMC), or ethylhydroxyethylcellulose (EHEC), as well as pluronics.

In another embodiment of the invention, the hydrogel formulation contains at least one pathway patency modulator, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, and EDTA.

In yet another embodiment of the invention, the hydrogel formulation includes at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline, and the mixtures thereof.

As noted above, in some embodiments of the invention, the reservoir comprises a solid coating that is disposed on at least one microprojection member of the delivery system. The coating formulation applied to the microprojection member to form the solid coating can comprise an aqueous and non-aqueous formulation having at least one antigenic agent, preferably, a vaccine, contained therein, which can be dissolved within a biocompatible carrier or suspended within the carrier.

In one embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-Cyclodextrin and sulfobutylether7 beta-cyclodextrin.

In one embodiment of the invention, the coating formulation includes at least one surfactant, which can be zwitterionic, amphoteric, cationic, anionic, or nonionic. Examples of suitable surfactants include sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan laurate, and alkoxylated alcohols, such as laureth-4.

In one embodiment of the invention, the concentration of the surfactant is in the range of approximately 0.001-2 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. % of the coating formulation.

In another embodiment, the coating formulation includes a hydrophilic polymer selected from the following group: poly(vinyl alcohol), poly(ethylene oxide), poly(2-hydroxyethylmethacrylate), poly(n-vinyl pyrolidone), polyethylene glycol and mixtures thereof, and similar polymers.

In a preferred embodiment, the concentration of the hydrophilic polymer in the coating formulation is in the range of approximately 0.01-20 wt. %.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In a further embodiment, the coating formulation includes a stabilizing agent, which can comprise, without limitation, a non-reducing sugar, a polysaccharide, a reducing or a DNase inhibitor.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating.

In yet another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, aspirin and EDTA.

In another embodiment of the invention, the coating formulation includes at least one antioxidant, which can be sequestering such as sodium citrate, citric acid, EDTA (ethylene-dinitrilo-tetraacetic acid) or free radical scavengers such as ascorbic acid, methionine, sodium ascorbate, and the like. Presently preferred antioxidants include EDTA and methionine.

In certain embodiments of the invention, the viscosity of the coating formulation is enhanced by adding low volatility counterions. In one embodiment, the agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

Another preferred embodiment is directed to a viscosity-enhancing mixture of counterions wherein the agent has a positive charge at the formulation pH and at least one of the counterion is an acid having at least two acidic pKas. The other counterion is an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Generally, in the noted embodiments of the invention, the amount of counterion should neutralize the charge of the antigenic agent. In such embodiments, the counterion or the mixture of counterion is present in amounts necessary to neutralize the charge present on the agent at the pH of the formulation. Excess of counterion (as the free acid or as a salt) can be added to the formulation in order to control pH and to provide adequate buffering capacity.

In another preferred embodiment, the agent has a positive charge and the counterion is a viscosity-enhancing mixture of counterions chosen from the group of citric acid, tartaric acid, malic acid, hydrochloric acid, glycolic acid, and acetic acid. Preferably, counterions are added to the formulation to achieve a viscosity in the range of about 20-200 cp.

In a preferred embodiment, the viscosity-enhancing counterion is an acidic counterion such as a low volatility weak acid. Low volatility weak acid counterions present at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment the counterion is a strong acid. Strong acids can be defined as presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions wherein at least one of the counterion is a strong acid and at least one of the counterion is a low volatility weak acid.

Another preferred embodiment is directed to a mixture of counterions wherein at least one of the counterions is a strong acid and at least one of the counterion is a weak acid with high volatility. Volatile weak acid counterions present at least one pKa higher than about 2 and a melting point lower than about 50° C. or a boiling point lower than about 170° C. at Pₐₜₘ. Examples of such acids include acetic acid, propionic acid, pentanoic acid and the like.

Preferably, the acidic counterion is present in amounts necessary to neutralize the positive charge present on the antigenic agent at the pH of the formulation. Excess of counterion (as the free acid or as a salt) can be added to the formulation in order to control pH and to provide adequate buffering capacity.

In yet other embodiments of the invention, particularly where the antigenic agent has a negative charge, the coating formulation further comprises a low volatility basic counter ion.

In a preferred embodiment, the coating formulation comprises a low volatility weak base counterion. Low volatility weak bases present at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, and glucosamine.

In another embodiment, the low volatility counterion comprises a basic zwitterions presenting at least one acidic pKa, and at least two basic pKa's, wherein the number of basic pKa's is greater than the number of acidic pkA's. Examples of such compounds include histidine, lysine, and arginine.

In yet other embodiments, the low volatility counterion comprises a strong base presenting at least one pKa higher than about 12. Examples of such bases include sodium hydroxide, potassium hydroxide, calcium hydroxide, and magnesium hydroxide.

Other preferred embodiments comprise a mixture of basic counterions comprising a strong base and a weak base with low volatility. Alternatively, suitable counterions include a strong base and a weak base with high volatility. High volatility bases present at least one basic pKa lower than about 12 and a melting point lower than about 50° C. or a boiling point lower than about 170° C. at Pₐₜₘ. Examples of such bases include ammonia and morpholine.

Preferably, the basic counterion is present in amounts necessary to neutralize the negative charge present on the antigenic agent at the pH of the formulation. Excess of counterion (as the free base or as a salt) can be added to the formulation in order to control pH and to provide adequate buffering capacity.

### BRIEF DESCRIPTION OF THE DRAWINGS

FIG. 1 is a partial perspective view of a microprojection array in accordance with the present invention;

FIG. 2 is a partial perspective view of a microprojection array having a solid antigen-containing coating on the microprojections; and

FIG. 3 is a side sectional view of an intradermal antigen delivery device useful in the present invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an antigenic agent” includes two or more such agents; reference to “a microprojection” includes two or more such microprojections and the like.

### Definitions

The terms “intradermal”, “intracutaneous”, “intradermally”, “intracutaneously”, “transdermal”, “transcutaneous”, “transdermally”, and “transcutaneously” are used interchangeably herein to mean that the antigenic agent is delivered into and/or through the skin into the epidermis layer and/or underlying dermis layer of the skin.

The term “transdermal flux”, as used herein, means the rate of transdermal delivery.

The terms “antigenic agent” and “vaccine” are used interchangeably herein and refer to a composition of matter or mixture containing an immunologically active agent or an agent, such as an antigen, which is capable of triggering a beneficial immune response when administered in an immunologically effective amount. The terms “antigenic agent” and “vaccine” thus include, without limitation, protein-based vaccines, polysaccharide-based vaccine, nucleic acid-based vaccines, viruses and bacteria.

Suitable antigenic agents that can be used in the present invention include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

A number of commercially available vaccines, which contain antigenic agents also have utility with the present invention including, without limitation, flu vaccines, lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukin 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitdis, pseudomonas aeruginosa, streptococcus pneumoniae, treponema pallidum, and vibrio cholerae, and mixtures thereof.

The noted vaccines can be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

It is to be understood that more than one antigenic agent may be incorporated into the agent source, reservoirs, and/or coatings of this invention, and that the use of the term “antigenic agent” in no way excludes the use of two or more such agents.

The term “biologically effective amount” or “biologically effective rate”, as used herein, means the antigenic agent is an immunologically active agent and refers to the amount or rate of the immunologically active agent needed to stimulate or initiate the desired immunologic, often beneficial result. The amount of the immunologically active agent employed in the hydrogel formulations and coatings of the invention will be that amount necessary to deliver an amount of the active agent needed to achieve the desired immunological result. In practice, this will vary widely depending upon the particular immunologically active agent being delivered, the site of delivery, and the dissolution and release kinetics for delivery of the antigenic agent or vaccine into skin tissues.

The term “microprojections”, as used herein, refers to piercing elements that are adapted to pierce or cut through the stratum corneum into the underlying epidermis layer, or epidermis and dermis layers, of the skin of a living animal, particularly a mammal and more particularly a human. In one embodiment of the invention, the microprojections have a projection length less than 1000 microns. In a further embodiment, the microprojections have a projection length of less than 500 microns, more preferably, less than 250 microns. The microprojections typically have a width and thickness of about 5 to 50 microns. The microprojections can also have a width of about 75 to 500 microns. The microprojections can be formed in different shapes, such as needles, hollow needles, blades, pins, punches, and combinations thereof. As such, the terms “microprojections,” “microprotrusions,” “microblades” and “microneedles” are used throughout interchangeably.

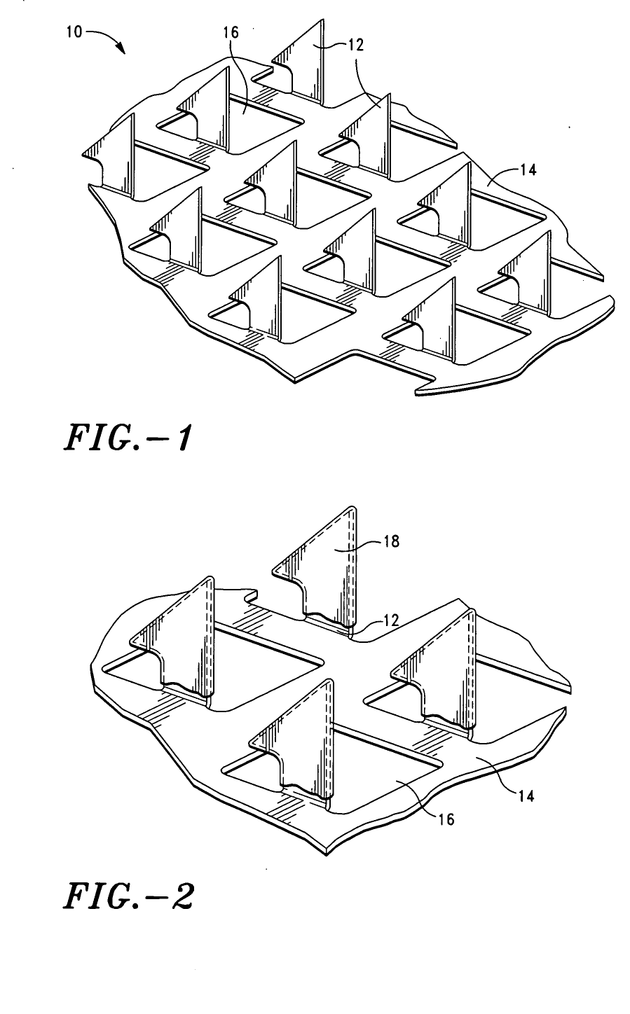

The terms “delivery member” and “microprojection member”, as used herein, generally connote a microprojection array comprising a plurality of microprojections arranged in an array for piercing the stratum corneum. The delivery member can be formed by etching or punching a plurality of microprojections from a thin sheet and folding or bending the microprojections out of the plane of the sheet to form a configuration such as that shown in FIG. 1 and described in Trautman et al., U.S. Pat. No. 6,083,196, which is hereby incorporated by reference in its entirety. The microprojection member can also be formed in other known manners, such as by forming one or more strips having microprojections along an edge of each of the strip(s), as disclosed in U.S. Pat. No. 6,050,988, which is hereby incorporated by reference in its entirety. Other microprojection arrays, and methods of making same, are disclosed in Godshall et al., U.S. Pat. No. 5,879,326 and Kamen, U.S. Pat. No. 5,983,136. The microprojection array can also comprise one or more hollow needles that hold a reservoir of dry pharmacologically active agent.

The present invention substantially reduces or eliminates the disadvantages and drawbacks associated with conventional methods for delivering an antigenic agent to a host (i.e., vaccination). As discussed in detail herein, the invention provides a unique two-step intradermal vaccination method for intradermally delivering an antigenic agent. The two-step intradermal vaccination method substantially reduces localized skin reactions (erythema and edema) at the skin sites where subsequent intradermal antigen applications are made.

Each delivery member includes a microprojection array having a plurality of stratum corneum-piercing microprojections extending therefrom and a reservoir containing the antigenic agent (e.g., a vaccine antigen) to be delivered. The reservoir is adapted and positioned to be in antigenic agent-transmitting relation to the slits cut through the stratum corneum by the piercing microprojections after application of the delivery member to the skin site.

In at least one embodiment, the reservoir comprises a distinct region of the delivery member that is disposed distal from but in communication with the microprojections, such as illustrated and described in U.S. Application Nos. 60/514,433 and 60/514,387; the disclosures of which are incorporated by reference herein in their entirety.

In one embodiment of the invention, the reservoir comprises a material (e.g., a polymeric gel material) in the form of a thin film laminated on the skin proximal or skin distal side of the microprojection array. Reservoirs of this type are disclosed in Theeuwes et al., WO 98/28037; the disclosure of which is incorporated by reference herein in its entirety.

In further embodiments of the invention, the reservoir comprises a biocompatible coating that is disposed on the delivery member, preferable, at least one microprojection thereof, more preferably, on the piercing tips of each microprojection. Typically, the microprojections have a length that allows skin penetration to a depth of less than about 400 microns, more preferably, less than about 300 microns. Upon piercing the stratum corneum layer of the skin, the antigenic agent contained in the reservoir is released into the skin for vaccination therapy.

Referring now to FIG. 1, there is shown one embodiment of stratum corneum-piercing microprojection member 10 for use with the present invention. FIG. 1 shows a portion of the member 10 having a plurality of microprojections 12. The microprojections 12 extend at substantially a 90° angle from a sheet 14 having openings 16. The member 10 may optionally be attached to a backing 22 having adhesive 24 for adhering the system 20 to the skin, as shown in FIG. 3.

In the embodiment of the microprojection member 10 shown in FIGS. 1, 2 and 3, the microprojections 12 are preferably formed by etching or punching a plurality of microprojections 12 from a thin metal sheet 14 and bending the microprojections 12 out of a plane of the sheet. Metals such as stainless steel and titanium are preferred. Metal microprojection members and methods of making same are disclosed in Trautman et al., U.S. Pat. No. 6,083,196; Zuck, U.S. Pat. No. 6,050,988; and Daddona et al., U.S. Pat. No. 6,091,975; the disclosures of which are incorporated by reference herein in their entirety.

Other microprojection members that can be used with the present invention are formed by etching silicon using silicon chip etching techniques or by molding plastic using etched micro-molds. Silicon and plastic microprojection members are disclosed in Godshall et al., U.S. Pat. No. 5,879,326; the disclosure of which is incorporated by reference herein.

According to the invention, the microprojection member 10 can be manufactured from various metals, such as stainless steel, titanium, nickel titanium alloys, or similar biocompatible materials. Preferably, the microprojection member 10 is manufactured out of titanium.

According to the invention, the microprojection member 10 can also be constructed out of a non-conductive material, such as a polymer. Alternatively, the microprojection member 10 can be coated with a non-conductive material, such as Parylene.

Microprojection members that can be employed with the present invention include, but are not limited to, the members disclosed in U.S. Pat. Nos. 6,083,196, 6,050,988 and 6,091,975; which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326; which is incorporated by reference herein in its entirety.

Suitable antigenic agents that can be delivered in accordance with the invention include, without limitation, vaccines, including protein-based vaccines, polysaccharide-based vaccine and nucleic acid-based vaccines, viruses and bacteria.

Further suitable antigenic agents include antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide)

Additional commercially available vaccines, which contain antigenic agents, include, without limitation, flu vaccines, lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)—N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; sclavo peptide: VQGEESNDK.HCl (IL-1β 163-171 peptide); and threonyl-MDP (Termurtide™): N-acetyl muramyl-L-threonyl-D-isoglutamine, and interleukin 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used. Other adjuvants include heat-shock proteins (HSPs); GTP-GDP; Loxoribine, MPL®; Murapalmitine; and Theramide™. Adjuvants are preferably non-irritating and non-sensitizing.

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitdis, pseudomonas aeruginosa, streptococcus pneumoniae, treponema pallidum, and vibrio cholerae, and mixtures thereof.

The noted antigenic agents or vaccines can be in various forms, such as free bases, acids, charged or uncharged molecules, components of molecular complexes or pharmaceutically acceptable salts. Further, simple derivatives of the active agents (such as ethers, esters, amides, etc.), which are easily hydrolyzed at body pH, enzymes, etc., can be employed.

As indicated, in accordance with one embodiment, the antigenic agent to be delivered can be contained in the hydrogel formulation. In the noted embodiment, the delivery member thus includes a hydrogel and means for receiving same (e.g., gel pack), such as disclosed in Co-Pending U.S. Patent Application Ser. No. 60/514,387, filed Oct. 24, 2003, 60/514,433, filed Oct. 24, 2003, 60/516,184, filed Oct. 31, 2003 and 60/524,062, filed Nov. 21, 2003; which are incorporated by reference herein in their entirety.

As indicated above, in at least one embodiment of the invention, the hydrogel formulation contains at least one antigenic agent. In an alternative embodiment of the invention, the hydrogel formulation is devoid of an antigenic agent and, hence, is merely a hydration mechanism.

According to the invention, when the hydrogel formulation is devoid of an antigenic agent, the antigenic agent is either coated on the microprojection 12, as described above, or contained in a solid film, such as disclosed in PCT Pub. No. WO 98/28037, which is similarly incorporated by reference herein in its entirety, on the skin side of the microprojection array, such as disclosed in the noted Co-Pending U.S. Patent Application Ser. No. 60/514,387, filed Oct. 24, 2003, or the top surface of the array. As discussed in detail in the noted Co-Pending Application, the solid film is typically made by casting a liquid formulation consisting of the antigenic agent, a polymeric material, such as hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), or pluronics, a plasticising agent, such as glycerol, propylene glycol, or polyethylene glycol, a surfactant, such as tween 20 or tween 80, and a volatile solvent, such as water, isopropanol, or ethanol. Following casting and subsequent evaporation of the solvent, a solid film is produced.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethyl-cellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. The noted polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection member 10 and skin and, optionally, the solid film.

According to the invention, adequate wetting properties are achieved by incorporating at least one wetting agent, such as a surfactant or polymer having amphiphilic properties, in the hydrogel formulation. Optionally, a wetting agent can also be incorporated in the solid film.

According to the invention, the surfactant can be zwitterionic, amphoteric, cationic, anionic, or nonionic. Examples of suitable surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

Suitable polymeric materials or polymers having amphiphilic properties include, without limitation, cellulose derivatives, such as hydroxyethylcellulose (HEC), hydroxypropyl-methylcellulose (HPMC), hydroxypropycellulose (HPC), methylcellulose (MC), hydroxyethyl-methylcellulose (HEMC), or ethylhydroxyethylcellulose (EHEC), as well as pluronics.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

According to the invention, the hydrogel formulation can include at least one pathway patency modulator or “anti-healing agent”, such as those disclosed in Co-Pending U.S. patent application Ser. No. 09/950,436, filed Sep. 8, 2001, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member 20. Examples of such agents include, without limitation, osmotic agents (e.g., sodium chloride), and zwitterionic compounds (e.g., amino acids).

The term pathway patency modulator or “anti-healing agent”, as defined in the noted Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextran sulfate sodium, and EDTA.

The hydrogel formulation can further include at least one vasoconstrictor, such as those disclosed in Co-Pending U.S. patent application Ser. Nos. 10/674,626, filed Sep. 29, 2003, and 60/514, filed Oct. 24, 2003, which are incorporated by reference herein in their entirety. As set forth in the noted Co-Pending Applications, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

According to the invention, the hydrogel formulation can also include a non-aqueous solvent, such as ethanol, propylene glycol, polyethylene glycol and the like, dyes, pigments, inert fillers, permeation enhancers, excipients, and other conventional components of pharmaceutical products or transdermal devices known in the art.

The hydrogel formulations of the invention exhibit adequate viscosity so that the formulation can be contained in a gel pack, keeps its integrity during the application process, and is fluid enough so that it can flow through the microprojection member openings and into the skin pathways.

For hydrogel formulations that exhibit Newtonian properties, the viscosity of the hydrogel formulation is preferably in the range of approximately 2-30 Poises (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

According to the invention, when the hydrogel formulation contains one of the aforementioned antigenic agents, the agent can be present at a concentration in excess of saturation or below saturation. The amount of an antigenic agent employed in the delivery system will be that amount necessary to deliver a therapeutically effective amount of the antigenic agent to achieve the desired result. In practice, this will vary widely depending upon the particular antigenic agent, the site of delivery, the severity of the condition, and the desired therapeutic effect. Thus, it is not practical to define a particular range for the therapeutically effective amount of an antigenic agent incorporated into the methods of the invention.

In one embodiment of the invention, the concentration of the antigenic agent is in the range of at least 1-40 wt. % of the hydrogel formulation.

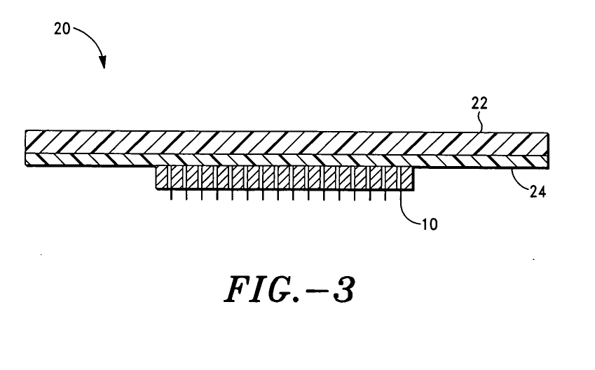

Referring now to FIG. 2, there is shown the microprojection member 10 having microprojections 12 having an antigen-containing reservoir 18 in the form of a solid coating 18 disposed on the microprojections 12. According to the invention, the coating 18 can partially or completely cover the microprojections 12.

The coating 18 can be applied to the microprojections 12 by dipping the microprojections 12 into a volatile liquid solution or suspension of the protein antigen and optionally any immune response augmenting adjuvant. The liquid solution or suspension should have an antigenic agent concentration of about 1 to 20 wt. %. The volatile liquid can be water, dimethyl sulfoxide, dimelthyl formamide, ethanol, isopropyl alcohol and mixtures thereof. Of these, water is most preferred.

According to the invention, the coating 18 can be applied to the microprojections 12 by a variety of known methods. Preferably, the coating 18 is only applied to those portions the microprojection member 10 or microprojections 12 that penetrate the skin.

The volatile liquid solution or suspension containing the antigenic agent can be applied to the microprojection array by immersion, spraying and/or other known microfluidic dispensing techniques. Preferably, only those portions of the microprojection array which penetrate into the skin tissue are coated with the antigenic agent. Suitable microprojection coatings and apparatus useful to apply such coatings are disclosed in U.S. patent application Ser. Nos. 10/045,842, filed Oct. 26, 2001, Ser. No. 10/099,604, filed Mar. 15, 2002, and 60/285,576; the disclosures of which are incorporated by reference herein.

Using the coating methods disclosed therein and the coating compositions disclosed herein, it is possible to precisely and uniformly coat only the tips of the skin piercing microprojections in typical metal (i.e., titanium) microprojection arrays. One such coating method comprises dip-coating. Dip-coating can be described as a means to coat the microprojections by partially or totally immersing the microprojections into a coating solution or formulation. By use of a partial immersion technique, it is possible to limit the coating to only the tips of the microprojections.

A further coating method comprises roller coating, which employs a roller coating mechanism, that similarly limits the coating to the tips of the microprojections. The roller coating method is disclosed in U.S. patent application Ser. No. 10/099,604, filed Mar. 15, 2002, which is incorporated by reference herein in its entirety. As discussed in detail in the noted application, the disclosed roller coating method provides a smooth coating that is not easily dislodged from the microprojections during skin piercing.

According to the invention, the microprojections can further include means adapted to receive and/or enhance the volume of the coating, such as apertures (not shown), grooves (not shown), surface irregularities (not shown) or similar modifications, wherein the means provides increased surface area upon which a greater amount of coating can be deposited.

Another coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 12. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

Furthermore, with microprojection tip coating, antigenic agent loadings of at least 0.2 micrograms per cm² of the microprojection array, more preferably, at least 2 micrograms per cm² of the array can readily be achieved. For a typical 5 cm² array, this translates into antigenic agent loadings of at least 1 microgram, and preferably at least 10 micrograms, which is adequate for most vaccinations.

With microprojection tip coating of the antigenic agent the antigenic agent delivery efficiency (E_{del}) is greatly enhanced; E_{del} being defined as the percent, by weight, of the antigenic agent released from the coating per predetermined period of time. With tip coating of the antigenic agent-containing solutions or suspensions of the present invention, E_{del} of at least 30% in 1 hour, and preferably at least 50% in 15 minutes can be achieved. Thus, the present invention offers significant cost advantages over conventional macrotine skin piercing devices used in the prior art.

As indicated, according to one embodiment of the invention, the coating formulations applied to the microprojection member 10 to form solid coatings can comprise aqueous and non-aqueous formulations having at least one antigenic agent disposed therein. According to the invention, the antigenic agent can be dissolved within a biocompatible carrier or suspended within the carrier.

According to the invention, the coating formulations preferably include at least one wetting agent, such as a surfactant and or polymer having amphiphilic properties. The surfactant(s) can be zwitterionic, amphoteric, cationic, anionic, or nonionic. Suitable surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols, such as laureth-4. Most preferred surfactants include Tween 20, Tween 80, and SDS.

Preferably, the concentration of the surfactant is in the range of approximately 0.001-2 wt. % of the coating formulation.

Suitable polymeric materials or polymers that have amphiphilic properties include, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. % of the coating formulation.

As will be appreciated by one having ordinary skill in the art, the noted wetting agents can be used separately or in combinations.

According to the invention, the coating formulation can further include a hydrophilic polymer. Preferably the hydrophilic polymer is selected from the following group: poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation is preferably in the range of approximately 0.01-20 wt. %.

According to the invention, the coating formulations can further include a biocompatible carrier such as those disclosed in Co-Pending U.S. patent application Ser. No. 10/127,108, filed Apr. 20, 2002, which is incorporated by reference herein in its entirety. Suitable biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation is preferably in the range of approximately 2-70 wt. %, more preferably, in the range of approximately 5-50 wt. % of the coating formulation.

The coating formulation and, hence, coating can further include a vasoconstrictor. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In yet another embodiment of the invention, the coating formulations include at least one “pathway patency modulator”. Suitable pathway patency modulators include, without limitation, osmotic agents (e.g., sodium chloride), zwitterionic compounds (e.g., amino acids) and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextran sulfate sodium, aspirin and EDTA.

According to the invention, the coating formulations can also include a non-aqueous solvent, such as ethanol, chloroform, ether, propylene glycol, polyethylene glycol and the like, dyes, pigments, inert fillers, permeation enhancers, excipients, and other conventional components of pharmaceutical products or transdermal devices known in the art.

In certain embodiments of the invention, the viscosity and stability of the antigenic agent containing coating formulation is enhanced by adding low volatility counterions. In one embodiment, the agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid.

Another preferred embodiment is directed to a viscosity-enhancing mixture of counterions wherein the agent has a positive charge at the formulation pH and at least one of the counterions is an acid having at least two acidic pKas. The other counterion is an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

Generally, in the noted embodiments of the invention, the amount of counterion should neutralize the charge of the antigenic agent. In such embodiments, the counterion or the mixture of counterion is present in amounts necessary to neutralize the charge present on the agent at the pH of the formulation. Excess of counterion (as the free acid or as a salt) can be added to the formulation in order to control pH and to provide adequate buffering capacity.

In one preferred embodiment, the agent has a positive charge and the counterion is a viscosity-enhancing mixture of counterions chosen from the group of citric acid, tartaric acid, malic acid, hydrochloric acid, glycolic acid, and acetic acid. Preferably, counterions are added to the formulation to achieve a viscosity in the range of about 20-200 cp.

In a preferred embodiment, the viscosity-enhancing counterion is an acidic counterion such as a low volatility weak acid. Low volatility weak acid counterions present at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment the counterion is a strong acid. Strong acids can be defined as presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions wherein at least one of the counterion is a strong acid and at least one of the counterion is a low volatility weak acid.

Another preferred embodiment is directed to a mixture of counterions wherein at least one of the counterion is a strong acid and at least one of the counterion is a weak acid with high volatility. Volatile weak acid counterions present at least one pKa higher than about 2 and a melting point lower than about 50° C. or a boiling point lower than about 170° C. at Pₐₜₘ. Examples of such acids include acetic acid, propionic acid, pentanoic acid and the like.

The acidic counterion is present in amounts necessary to neutralize the positive charge present on the agent at the pH of the formulation. Excess of counterion (as the free acid or as a salt) can be added to the formulation in order to control pH and to provide adequate buffering capacity.

In yet other embodiments of the invention, particularly where the antigenic agent has a negative charge, the coating formulation further comprises a low volatility basic counter ion.

In a preferred embodiment, the coating formulation comprises a low volatility weak base counterion. Low volatility weak bases present at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, and glucosamine.

In another embodiment, the low volatility counterion comprises a basic zwitterion presenting at least one acidic pKa, and at least two basic pKa's, wherein the number of basic pKa's is greater than the number of acidic pkA's. Examples of such compounds include histidine, lysine, and arginine.

In yet other embodiments, the low volatility counterion comprises a strong base presenting at least one pKa higher than about 12. Examples of such bases include sodium hydroxide, potassium hydroxide, calcium hydroxide, and magnesium hydroxide.

Other preferred embodiments comprise a mixture of basic counterions comprising a strong base and a weak base with low volatility. Alternatively, suitable counterions include a strong base and a weak base with high volatility. High volatility bases present at least one basic pKa lower than about 12 and a melting point lower than about 50° C. or a boiling point lower than about 170° C. at Pₐₜₘ. Examples of such bases include ammonia and morpholine.

Preferably, the basic counterion is present in amounts necessary to neutralize the negative charge present on the antigenic agent at the pH of the formulation. Excess of counterion (as the free base or as a salt) can be added to the formulation in order to control pH and to provide adequate buffering capacity.

Further discussion regarding the use of low volatility counterions can be found in U.S. Patent Application Ser. No. 60/484,020, filed Jun. 30, 2003 and 60/484,020, filed Jun. 30, 2003; the disclosures of which are incorporated by reference herein in their entirety.

In another embodiment of the invention, the coating formulation includes at least one buffer. Examples of suitable buffers include ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, b-hydroxybutyric acid, crotonic acid, angelic acid, hydracrylic acid, aspartic acid, glutamic acid, glycine or mixtures thereof.

In one embodiment of the invention, the coating formulation includes at least one antioxidant, which can be sequestering such sodium citrate, citric acid, EDTA (ethylene-dinitrilo-tetraacetic acid) or free radical scavengers such as ascorbic acid, methionine, sodium ascorbate, and the like. Presently preferred antioxidants include EDTA and methionine.

In the noted embodiments of the invention, the concentration of the antioxidant is in the range of approximately 0.01-20 wt. % of the coating formulation.

Other known formulation additives can also be added to the coating formulations as long as they do not adversely affect the necessary solubility and viscosity characteristics of the coating formulation and the physical integrity of the dried coating.

Preferably, the coating formulations have a viscosity less than approximately 500 centipoise and greater than 3 centipoise in order to effectively coat each microprojection 10. More preferably, the coating formulations have a viscosity in the range of approximately 3-200 centipoise.

According to the invention, the desired coating thickness is dependent upon the density of the microprojections per unit area of the sheet and the viscosity and concentration of the coating composition as well as the coating method chosen. Preferably, the coating thickness is less than 50 microns.

In one embodiment, the coating thickness is less than 25 microns, more preferably, less than 10 microns as measured from the microprojection surface. Even more preferably, the coating thickness is in the range of approximately 1 to 10 microns.

In all cases, after a coating has been applied, the coating formulation is dried onto the microprojections 12 by various means. In a preferred embodiment of the invention, the coated member is dried in ambient room conditions. However, various temperatures and humidity levels can be used to dry the coating formulation onto the microprojections. Additionally, the coated member can be heated, lyophilized, freeze dried or similar techniques used to remove the water from the coating.

The microprojection member 10 is preferably suspended in a retainer ring as described in detail in Co-Pending U.S. patent application Ser. No. 09/976,762, filed Oct. 12, 2001, which is incorporated by reference herein in its entirety. After placement of the microprojection member 10 in the retainer ring, the microprojection member 10 is applied to the patient's skin, preferably with an impact applicator, such as disclosed in Co-Pending U.S. patent application Ser. No. 09/976,798, filed Oct. 12, 2001, which is incorporated by reference herein in its entirety.

### EXAMPLE 1

This example investigates whether boosting with a lower dose minimizes the skin response while providing an adequate immune response. The general regimen consists of intradermally administering a large dose of the vaccine during the primary immunization followed by one or more intradermal booster immunizations with lower doses of the vaccine.

Experiments have demonstrated that up to 80 micrograms ovalbumin was delivered over the 1 hour application period. Bolus delivery (5 seconds application) resulted in about 25 micrograms delivered. These experiments further demonstrated that delivery of ovalbumin could be controlled by adjusting the amount of ovalbumin on the array.

Based on these results, two immunization regimens are effective for reducing the skin response. The first regimen involves administering the primary immunization and booster administration with identical coated microprojection arrays. However, the wearing time during the primary induction immunization is longer than the wearing time during booster immunization. For example, primary immunization administration can be performed for as long as 24 hours. Booster immunization administration can be as long as 30 minutes, preferably less than 15 minutes. These administration periods effect delivery of a large dose of the vaccine during the primary immunization. Subsequently, lower doses of the vaccine are administered during the booster immunizations.

The second regimen involves administering the primary immunization and booster administration with different microprojection arrays. The wearing times during the primary immunization and the booster administration are identical. In practice, the primary immunization is performed with the system delivering the largest dose of the vaccine, for example a microprojection array having a high antigen concentration coating. Subsequently, booster immunizations are performed with the system delivering a lower dose of the vaccine, for example, a microprojection array having a low antigen concentration coating. Wearing time could be as long as 30 minutes, preferably as long as 15 minutes. Alternatively, adjusting the microprojection density or skin contact area can also effectively reduce the amount of antigen delivered for the booster administration.

The method of the present invention allows convenient intradermal vaccination therapy while avoiding undesirable skin reactions, and is broadly applicable to intracutaneous delivery of a wide variety of therapeutic vaccines to improve efficacy and provide convenience.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method for delivering an antigenic agent to a mammal, comprising:
providing at least two transdermal delivery members, each of said members including a plurality of microprojections configured to pierce the stratum corneum and a reservoir containing a loading amount of said antigenic agent, said reservoir being adapted to be positioned in antigenic agent-transmitting relation with the mammal when the delivery member is applied to a skin site of the mammal;
delivering with a first of said at least two transdermal delivery members an induction amount of said antigenic agent;
delivering with a second of said at least two transdermal delivery members a first booster amount of said antigenic agent at least about 7 days after said delivery of said induction amount of said antigenic agent, said booster amount comprising up to about 50% by weight of said induction amount.

2. The method of claim 1, wherein said induction amount of said antigenic agent is at least about 10 micrograms and said first booster amount of said antigenic agent is below about 5 micrograms.

3. The method of claim 1, wherein said first booster amount of said antigenic agent is delivered at least 14 days after said step of delivering said induction amount of said antigenic agent.

4. The method of claim 1, wherein said loading amount of said antigenic agent is substantially the same in said first and second transdermal delivery members, wherein said step of delivering said induction amount of said antigenic agent comprises leaving said first transdermal delivery member in contact with said mammal for a first period of time and said step of delivering said first booster amount of said antigenic agent comprises than leaving said second transdermal delivery member in contact with said mammal for a second period of time and wherein said first period of time is longer than said second period of time.

5. The method of claim 4, wherein said first period of time is at least about 0.5 hours.

6. The method of claim 5, wherein said second period of time is less than about 0.25 hours.

7. The method of claim 1, wherein said first transdermal delivery member has a loading amount of said antigenic agent greater than the loading amount of said antigenic agent of said second transdermal delivery member.

8. The method of claim 7, wherein said first delivery member is left in skin piercing contact with the mammal for about the same period of time as said second delivery member.

9. The method of claim 1, including delivering a second booster amount of said antigenic agent with a third transdermal delivery member at least about 7 days following said step of delivering said first booster amount of said antigenic agent.

10. The method of claim 1, wherein said first and second transdermal delivery members are comprised of metal and include an adhesive backing.

11. The method of claim 1, wherein said first and second transdermal delivery members pierce the skin over a skin contact area of less than 5 cm².

12. The method of claim 1, further comprising the step of substantially reducing local skin reactions to said antigenic agent.

13. The method of claim 1, wherein said antigenic agent is selected from the group consisting of proteins, polysaccharide conjugates, oligosaccharides, lipoproteins, subunit vaccines, Bordetella pertussis (recombinant PT accince—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diptheriae, group A streptococcus, legionella pneumophila, neisseria meningitdis, pseudomonas aeruginosa, streptococcus pneumoniae, treponema pallidum, vibrio cholerae, flu vaccines, lyme disease vaccine, rabies vaccine, measles vaccine, mumps vaccine, chicken pox vaccine, small pox vaccine, hepatitis vaccine, pertussis vaccine, diptheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

14. The method of claim 1, wherein the reservoir includes an immunologically potentiating adjuvant.

15. The method of claim 14, wherein said adjuvant is selected from the group consisting of aluminum phosphate gel, aluminum hydroxide, algal glucan, β-glucan, cholera toxin B subunit, CRL1005, ABA block polymer with mean values of x=8 and y=205, gamma insulin, linear (unbranched) β-D(2->1) polyfructofuranoxyl-α-D-glucose, Gerbu adjuvant, N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C₅₉H₁₀₈N₆O₁₉PNa-3H₂O (MTP), Murametide, Nac-Mur-L-Ala-D-Gln-OCH₃, Pleuran, β-glucan, QS-21; S-28463, 4-amino-a, a-dimethyl-1H-imidazo[4,5-c]quinoline-1-ethanol, sclavo peptide, VQGEESNDK.HCl (IL-1β 163-171 peptide), threonyl-MDP (Termurtide™), N-acetyl muramyl-L-threonyl-D-isoglutamine, interleukin 18, IL-2 IL-12, IL-15, DNA oligonucleotides, CpG containing oligonucleotides, gamma interferon, NF kappa B regulatory signaling proteins, heat-shock proteins (HSPs), GTP-GDP, Loxoribine, MPL®, Murapalmitine, and Theramide™.

16. The method of claim 1, wherein said reservoir includes a hydrogel formulation.

17. The method of claim 16, wherein said hydrogel formulation comprises a macromolecular polymeric network.

18. The method of claim 17, wherein said macromolecular polymeric network is selected from the group consisting of hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), and pluronics.

19. The method of claim 1, wherein said reservoir comprises a coating disposed on at least one of said first and second delivery members.

20. The method of claim 19, wherein said coating further includes a low volatility counterion.

21. The method of claim 20, wherein said low volatility counterion is selected from the group consisting of maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid, and phosphoric acid, and mixtures thereof.

22. The method of claim 20, wherein said low volatility counterion is selected from the group consisting of monoethanolomine, diethanolamine, triethanolamine, tromethamine, methylglucamine, glucosamine, histidine, lysine, arginine, sodium hydroxide, potassium hydroxide, calcium hydroxide, magnesium hydroxide, ammonia and morpholine, and mixtures thereof.

23. The method of claim 1, wherein said reservoir includes a surfactant.

24. The method of claim 23, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

25. The method of claim 1, wherein said reservoir includes an amphiphilic polymer.

26. The method of claim 25, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

27. The method of claim 1, wherein said reservoir includes a pathway patency modulator.

28. The method of claim 27, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

29. The method of claim 1, wherein said reservoir includes a vasoconstrictor.

30. The method of claim 29, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

31. The method of claim 1, wherein said reservoir includes an antioxidant.

32. The method of claim 31, wherein said antioxidant is selected from the group consisting of sodium citrate, citric acid, ethylene-dinitrilo-tetraacetic acid (EDTA), ascorbic acid, methionine, and sodium ascorbate.

33. The method of claim 1, wherein said reservoir includes a solubilising/complexing agent.

34. The method of claim 33, wherein said solubilising/complexing agent is selected from the group consisting of Alpha-Cyclodextrin, Beta-Cyclodextrin, Gamma-Cyclodextrin, glucose-alpha-Cyclodextrin, maltosyl-alpha-Cyclodextrin, glucosyl-beta-Cyclodextrin, maltosyl-beta-Cyclodextrin, hydroxypropyl beta-cyclodextrin, 2-hydroxypropyl-beta-Cyclodextrin, 2-hydroxypropyl-gamma-Cyclodextrin, hydroxyethyl-beta-Cyclodextrin, methyl-beta-Cycl odextrin, sulfobutylether-alpha-cyclodextrin, sulfobutylether-beta-cyclodextrin, and sulfobutylether-gamma-cyclodextrin.

35. The method of claim 1, wherein said mammal comprises a human.

36. A method for vaccinating a mammal, comprising:
providing at least two transdermal delivery members, each of said members comprising at least one microprojection configured to pierce the stratum corneum and a reservoir having a loading amount of an antigenic agent, said reservoir being positioned in antigenic agent-transmitting relation with said mammal;
delivering with a first of said at least two transdermal delivery members an induction amount of said antigenic agent;
delivering with a second of said at least two transdermal delivery members a booster amount of said antigenic agent at least about 7 days thereafter, said booster amount being up to about 50% by weight of said induction amount.

37. A method for vaccinating a mammal, comprising:
providing at least two transdermal delivery members, each of said members comprising at least one microprojection configured to pierce the stratum corneum and a reservoir having a loading amount of an antigenic agent, said reservoir being positioned in antigenic agent-transmitting relation with said mammal;
delivering with a first of said at least two transdermal delivery members an induction amount of said antigenic agent;
delivering with a second of said at least two transdermal delivery members a booster amount of said antigenic agent, said booster amount being up to about 50% by weight of said induction amount.
